(19) United States
(12) Patent Application Publication
(10) Pub. No.: US 2016/0262678 A1
(43) Pub. Date: Sep. 15, 2016

(54) RECEPTACLE DEVICE, METHOD FOR PROVIDING THE SAME AND METHOD FOR SEPARATING A MIXTURE

(71) Applicant: GREINER BIO-ONE GMBH, Kremsmünster (AT)

(72) Inventor: Christian BAUER, Kremsmuenster (AT)

(73) Assignee: Greiner Bio-One GmbH, Kremsmuenster (AT)

(21) Appl. No.: 15/036,483

(22) PCT Filed: Nov. 13, 2014
(86) PCT No.: PCT/AT2014/050273

§ 371 Date: May 13, 2016

(30) Foreign Application Priority Data
Nov. 14, 2013 (AT) A 50759/2013

(51) Int. Cl. A61B 5/15; B01D 21/26; A61B 10/00; A61B 5/154; B01L 3/00; G01N 33/49

(52) U.S. Cl. CPC A61B 5/150755; B01L 3/50215; G01N 33/491; A61B 10/0096; A61B 5/154; B01D 21/262; B01L 2400/0633; B04B 5/0414

(57) ABSTRACT

The invention relates to a receptacle device (1) for separating a mixture (2) into a lighter phase (3) and a heavier phase (4), which comprises a receptacle (5) having an open end (7) and an end (8) closed by a base (9), and the open end (7) is closed by a removable closure unit (15). A separating element (14) made of an elastically deformable material is arranged in a receptacle chamber (13), and the separating element (14) in its non-deformed initial state has a spherical shape and is arranged in the region of the base (9) and contacts an internal face (11) of a side wall (10) in a contact plane (23) oriented perpendicular to a longitudinal axis (6). An external diameter (24) of the separating element (14) is selected to be larger in the non-deformed initial state thereof than a cross-sectional dimension (25) of the receptacle chamber (13) in the contact plane (23). The separating element (14) is held positioned on the internal face (11) of the side wall (10) in a pre-tensioned standby position. The invention further relates to a method for providing a receptacle device (1) of this type and a method for separating a mixture (2) into a lighter phase (3) and a heavier phase (4).

The invention relates to a receptacle device for separating a mixture, in particular blood, as described in claim 1. However, this invention further relates to a method for providing a receptacle device of this type and a method for separating a mixture, in particular blood, into its lighter phase having a lower density and its heavier phase having a relatively higher density, as described in claims 13 and 18.

U.S. Pat. No. 3,508,653 A respectively DE 1 806 196 A disclose a receptacle device for separating fluids, in particular blood, into its lighter phase having a lower density and its heavier phase with a relatively higher density. The receptacle device comprises a receptacle having first and second ends spaced apart from one another in the direction of a longitudinal axis, the first end being open and the second end being closed by a base. Extending between the first and second ends is a side wall with an internal face and an external face, and the side wall and base bound a receptacle chamber. Provided in the receptacle chamber as a separating element is a plunger, which is made entirely from an elastically deformable material. The open end of the receptacle is closed by a removable closure unit. Before the start of separation, the plunger is releasably held in position on a seal element of the closure unit.

Another separating device for separating fluids, in particular blood, into its lighter phase having a lower density and its heavier phase with a relatively higher density is known from JP 56-168847 A. In this instance, a bladder made from a thin film and of a spherical shape is provided in the receptacle as a separating element, the interior of which is filled with a gel.

EP 1 005 909 B1 describes an assembly for separating a fluid sample into a phase with a higher relative density and a phase with a lower relative density. The receptacle device comprises a tube with an open end and an end closed by a base. Extending between the two ends is a side wall with an internal face and an external face. The open end of the tube is closed by means of a closure. An elastically deformable liner is disposed in the tube, which is configured so that it can be elastically widened. Also disposed in the receptacle chamber is a spherical seal body which, when the liner is in the non-widened state, lies against the latter. The seal body is made from a rigid, thermoplastic material. During the separation process, the liner is elastically deformed, as a result of which the seal body is released and due to its selected density floats between the two phases to be separated on the phase with the higher density.

Other separating devices are known in which the separating element is provided or coated with a material which swells on contact with liquid. Such an example is described in EP 0 744 026 B1 respectively DE 695 24 063 T2. These are expensive to manufacture and are not sufficiently reliable in terms of the separation result.

The underlying objective of this invention is to propose a receptacle device which is inexpensive to manufacture and requires few components in spite of guaranteeing a reliable separation of the phases being separated from one another. Furthermore, a method for providing such a receptacle device is specified, which enables simple and inexpensive manufacture. Also specified is a method for separating a mixture using a receptacle device of this type, which ensures perfect separation of the phases to be separated from one another, even after a longer period of storage.

One objective of the invention is achieved by the features defined in claim 1. The advantage obtained as a result of the features defined in claim 1 is that by opting for the spherical shape of the separating element, adhesion of constituents of the phases to be separated is made more difficult or prevented altogether because of the spherical surface. The fact that the separating element is disposed in the base region of the tubular receptacle means that standard sampling devices can be used when taking the blood sample, for example. When filling with the mixture, in particular blood, the seal element of the closure unit merely has to be pierced and the separating element is unaffected by this. Filling can therefore proceed unobstructed in a known manner. Disposing the separating element in the base region also means that the standard method can be used to manufacture the receptacle device, such as used to produce standardized sample-taking and receptacle devices used for taking blood samples. The fact that the separating element is pre-positioned and held clamped in its standby position means that a defined position of the separating element is guaranteed to be maintained inside the receptacle chamber during transport and up until the filling operation. Choosing an elastically deformable material for the separating element also means that additional components inside the receptacle can be dispensed with. Not only does this result in a reliable standby position, a perfect durable separation is also obtained on completion of the separation operation. Due to the pre-positioned standby position, a sufficient filling volume is always guaranteed so that the receptacle chamber can be filled with a sufficient quantity of the sample.

Another embodiment defined in claim 2 is of advantage because being in the standby position already and as a function of the dimensional differences, the separating element can be guaranteed to remain in a relative fixed position inside the receptacle chamber even in the event of temperature fluctuations and impacts.

An embodiment defined in claim 3 is also of advantage because a deformation of the separating element can be obtained that is sufficient to form the flow passage between the separating element and the side wall of the receptacle. Furthermore, due to the choice of material, an integral, one-part component is obtained, from which material particles can be prevented from becoming detached, and not just when centrifuging. Contamination of the contained sample due to detached particles of the separating element such as can very easily occur if using a gel, for example, can therefore be prevented.

Due to the embodiment defined in claim 4, it is possible to guarantee that the separating element will float reliably at the separation plane or interface between the two phases to be separated.

Based on another embodiment defined in claim 5, if blood in particular is the mixture to be separated, it can be more easily introduced into the receptacle chamber. Furthermore, however, the filling quantity and hence the volume of the sample can be set depending on the selected negative pressure.

Another embodiment defined in claim 6 or 7 is of advantage because a better seal seat can be obtained for the sealing stopper to be inserted in the receptacle chamber in the region of the open end.

Due to the embodiment defined in claim 8, starting from the standby position of the separating element inside the receptacle chamber as well as in the separating position subsequently assumed, a perfect and sealed separation of the two part chambers of the receptacle chamber on either side of the separating element in the axial direction can be guaranteed. A perfect and durable separation of the two mutually separated media or phases of the mixture can therefore also be obtained in the separating position.

As a result of the embodiment defined in claim 9, a range of different filling quantities of the mixture can be separated. In addition, reliable sealing contact of the separating element in its separating position can always be obtained across a bigger axial extension.

Also of advantage is an embodiment defined in claim 10 because it enables easier insertion of the separating element in the receptacle chamber. As a result, the separating element is not positioned in the sealing contact with the internal face until reaching the region of the second part section. Furthermore, manufacture is also made easier, especially the demolding operation if using an injection casting process.

Based on an embodiment such as that defined in claim 11, deposits of heavier constituents of the mixture can be prevented during the separation operation. This enables subsequent contamination of the lighter phase of the mixture after centrifugation to be prevented.

In this respect, an embodiment defined in claim 12 has proved to be of advantage because even when the separating element has been inserted in the region of the base, the part chamber of the receptacle chamber formed between the separating element and base together with the part chamber disposed above it can be reduced to a pressure below atmospheric pressure. This makes assembly and preparation of the receptacle device easier and also results in a longer period of storage without any detrimental change in the negative pressure.

The objective of the invention can also be achieved independently using an approach based on the features defined in claim 13. The advantages obtained as a result of this combination of features are that by opting for the spherical shape of the separating element, adhesion of constituents of the phases to be separated is made more difficult or prevented altogether because of the spherical surface. The fact that the separating element is disposed in the base region of the tubular receptacle means that standard sampling devices can be used when taking the blood sample, for example. When filling with the mixture, in particular blood, the seal element of the closure unit merely has to be pierced and the separating element is unaffected by this. Filling can therefore proceed unobstructed in a known manner. Disposing the separating element in the base region also means that the standard method can be used to manufacture the receptacle device, such as used to produce standardized sample-taking and receptacle devices used for taking blood samples. The fact that the separating element is pre-positioned and held clamped in its standby position means that a defined position of the separating element is guaranteed to be maintained inside the receptacle chamber during transport and up until the filling operation. Choosing an elastically deformable material for the separating element also means that additional components inside the receptacle can be dispensed with. Not only does this result in a reliable standby position, a perfect durable separation is also obtained on completion of the separation operation. Due to the pre-positioned standby position, a sufficient filling volume is always guaranteed so that the receptacle chamber can be filled with a sufficient quantity of the sample.

An approach based on the features defined in claim 14 is also of advantage because by simply deforming the separating element and placing it inside the fitting tube, it can be moved to the base region of the receptacle and it is not until then than it is positioned in the predefined standby position. This even means that prior evacuation of the receptacle chamber could be dispensed with because based on an appropriate choice of the dimensions of the fitting tube and a sufficient pre-deformation of the separating element, the latter can firstly be moved into contact with the base wall of the base as it is pushed out of the fitting tube before being completely pushed out of it. This prevents any undesired inclusion of ambient air between the separating element and base.

Another advantageous approach is defined in claim 15, whereby although it is necessary to pierce or puncture the separating element with the assembly pin, the separating element can be disposed in the base region of the receptacle in such a way that the inclusion of air and residual air quantities can be reliably prevented.

A variant of the method defined in claim 16 is of advantage because, again with this approach, the inclusion of a residual quantity of air between the separating element and base can be prevented. This avoids any detrimental effect on storage life because the receptacle chamber is evacuated and there can be no further drop in the negative pressure caused by an inclusion of air.

Another approach based on the features defined in claim 17 is of advantage, because if blood in particular is the mixture to be separated, it can be more easily introduced into the receptacle chamber. Furthermore, however, the filling quantity and hence the volume of the sample can be set depending on the selected negative pressure.

The objective of the invention can also be achieved independently on the basis of a method for separating a mixture, in particular blood, incorporating the features defined in claim 18. The advantages obtained as a result of this combination of features are that using the receptacle device based on the design proposed by the invention and the separating element disposed in it, a standardized centrifugation process can be run. On completion of this process, due to the fact that only an elastic deformation of the separating element takes place, a reliable sealing position is obtained in its separating position. By opting for the spherical shape in conjunction with the elastic material, however, parts of the separating element are prevented from becoming detached, unlike the situation of using a gel when this poses a major risk.

Finally, another advantageous approach is defined in claim 19, whereby even the smallest of constituents with a higher density can be reliably moved into the part chamber formed between the separating element and the base of the receptacle. This results in a very high degree of purity of the lighter phase moved between the separating element and the open end of the receptacle.

To provide a clearer understanding, the invention will be described in more detail below with reference to the appended drawings.

These are highly simplified, schematic diagrams illustrating the following:

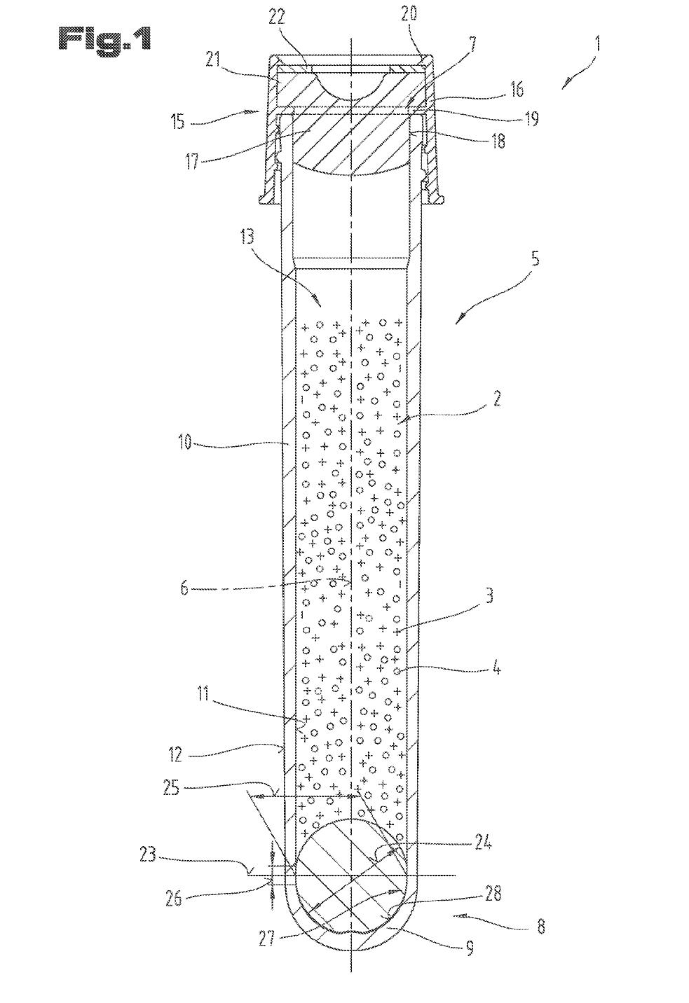

FIG. 1 a receptacle device filled with a mixture prior to the start of the separation operation, viewed in axial section;

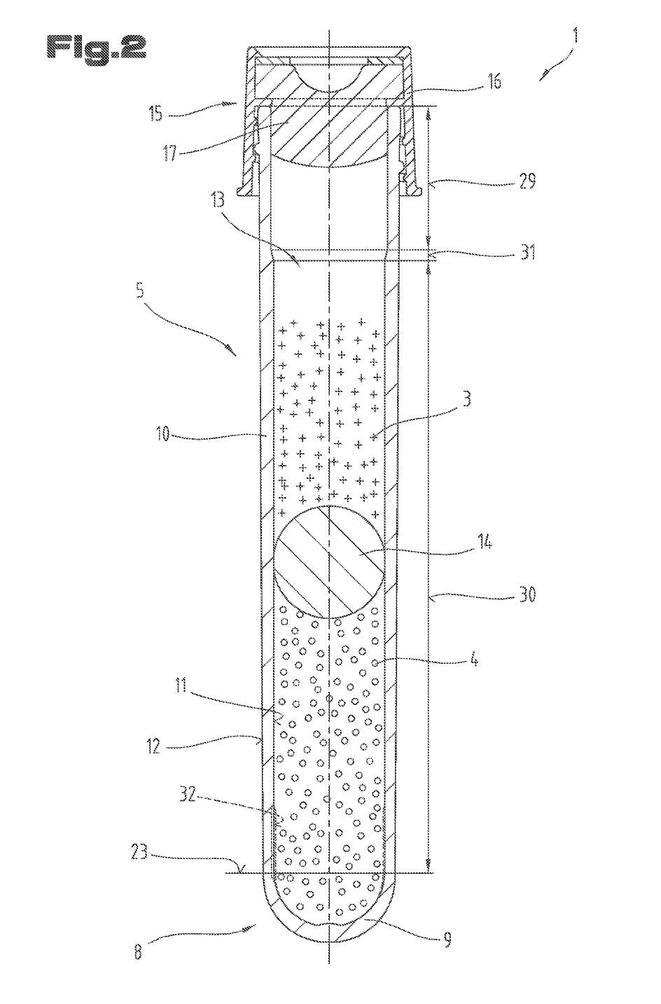

FIG. 2 the receptacle device illustrated in FIG. 1, having completed the separation operation;

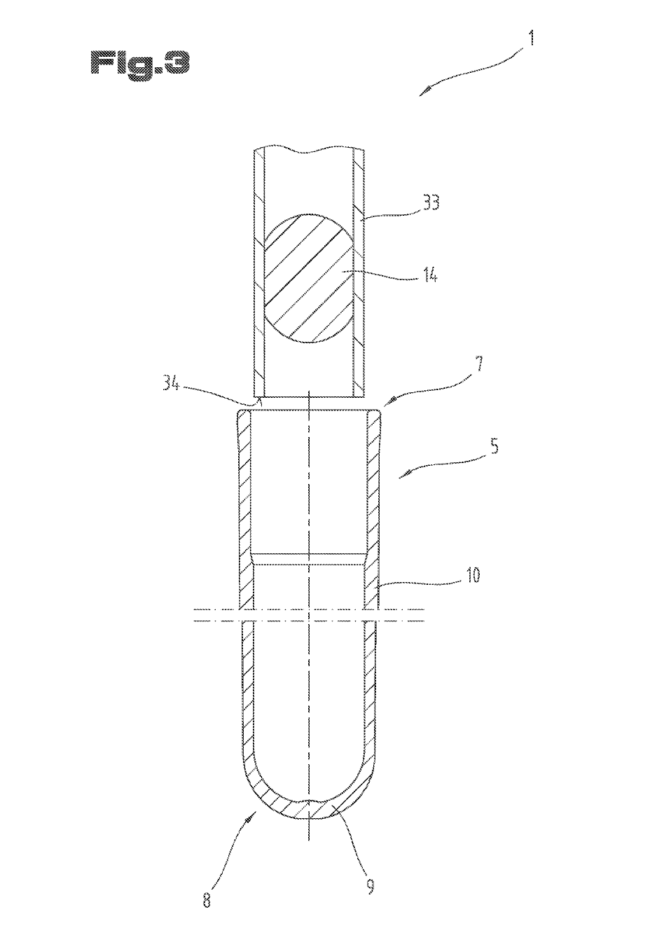

FIG. 3 a first assembly option for introducing the separating element into the receptacle chamber of the receptacle;

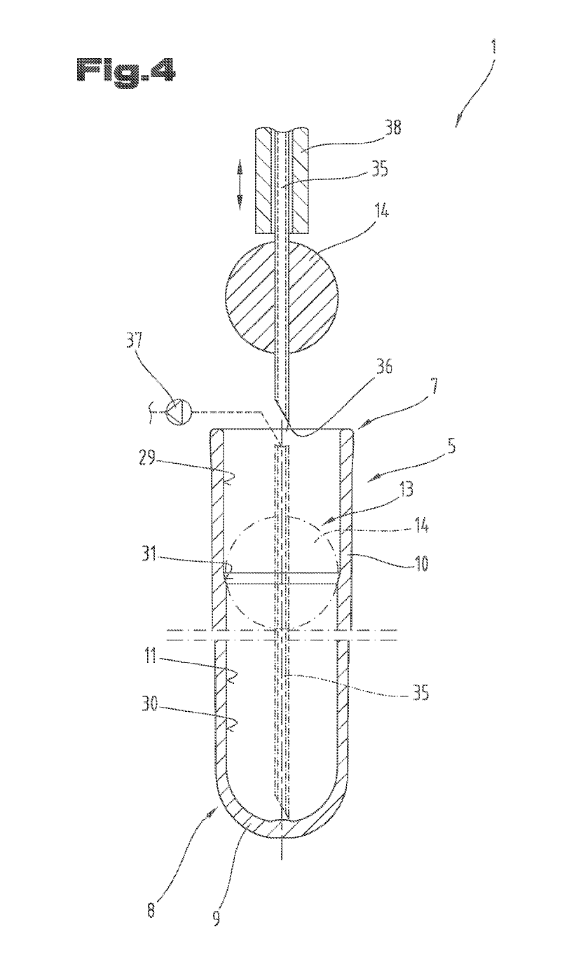

FIG. 4 a second assembly option for introducing the separating element into the receptacle chamber of the receptacle;

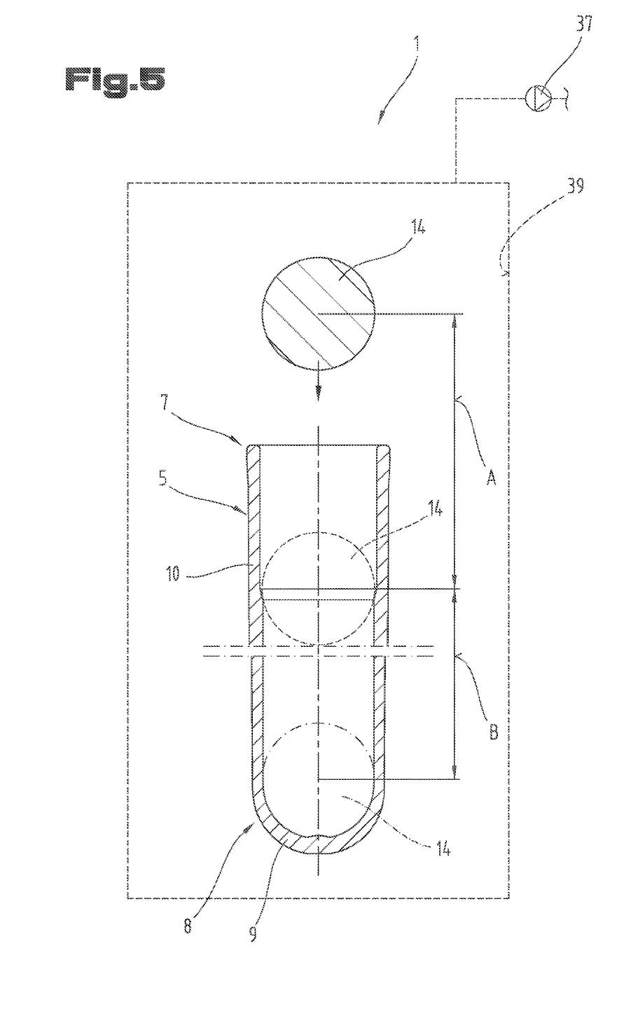

FIG. 5 a third assembly option for introducing the separating element into the receptacle chamber of the receptacle.

Firstly, it should be pointed out that the same parts described in the different embodiments are denoted by the same reference numbers and the same component names and the disclosures made throughout the description can be transposed in terms of meaning to same parts bearing the same reference numbers or same component names. Furthermore, the positions chosen for the purposes of the description, such as top, bottom, side, etc., relate to the drawing specifically being described and can be transposed in terms of meaning to a new position when another position is being described.

FIGS. 1 and 2 illustrate a receptacle device 1 for a mixture 2 or substance made up of at least two different constituents or media, such as body fluids, tissue parts or tissue cultures, for example, which is designed to enable the mixture 2 contained in the receptacle device 1 to be separated into at least two of its constituents. This is usually done mechanically by applying centrifugal force. For separating or separation purposes, the mixture 2 is preferably in a liquid aggregate state.

This receptacle device 1 is preferably used for separating blood, one element of which will be referred to here as the lighter phase 3 with a lower density and another as the heavier phase 4 with a relatively higher density. In the case of blood, the lighter phase 3 is serum or plasma, for example, and the heavy phase is the cellular component, such as erythrocytes, leucocytes and thrombocytes, for example.

This separation or splitting of the mixture 2 into its two phases 3, 4 may be done in a conventional way, physically, by centrifugation. For example, this is run starting from the nonoperating position until a radial centrifugal acceleration of 1,000 g to 5,000 g, preferably between 1,800 g and 3,500 g is reached, where “g” denotes the acceleration of gravity and the value of 1 g=9.81 m/s². As a result, it is possible to separate the lighter phase 3 from the heavier, usually more solid phase 4 on the basis of the different density values, as will be described in more detail below with reference to the drawings.

The receptacle device 1 comprises an approximately cylindrical, usually tubular, receptacle 5, having first and second ends 7, 8 spaced apart from one another in the direction of a longitudinal axis 6. Based on this example of an embodiment, the first end 7 is open and the second end 8 is closed by a base 9. The receptacle 5 further comprises a side wall 10 or container wall extending between the first and second ends 7, 8 and in turn having an internal face 11 and an external face 12. The side wall 10 and the base 9 also bound a receptacle chamber 13 into which the mixture to be separated 2 is introduced or drawn.

The receptacle device 1 further comprises a separating element 14 which is disposed inside or in the receptacle chamber 13 and which is preferably made entirely or completely from an elastically deformable material. The material selected for the separating element 14 may be a material which is self-closing after being pierced. This means that after removing the piercing element, the material in the region of the separation point or piercing point elastically rebounds of its own accord to the extent that the separating element 14 is gas-tight and/or liquid-proof. Elastically deformable materials which may be used include, for example, thermoplastic elastomers (TPE), silicone, rubber, pharmaceutical rubber, silicone rubber or such like.

The receptacle device 1 may also comprise a removable closure unit 15 which in this particular instance closes the first, open end 7 of the receptacle 5.

The closure unit 15 may in turn be based on various designs and in the case of the embodiment illustrated as an example here comprises a cap 16 surrounding the first end 7 and a seal element 17 retained in it, for example a sealing plug. The seal element 17 is usually a highly elastic and self-closing material which can be pierced, e.g. pharmaceutical rubber, silicone rubber or bromobutyl rubber. The part of the seal element 17 that is inserted in the first open end 7 has a sealing surface 18 as viewed in the axial direction, which sits in a sealing arrangement on the internal face 11. This enables the receptacle 5 to be sealed in a known manner.

The cap 16, in particular its cap side wall, is disposed concentrically with the longitudinal axis 6 and is of a circular or approximately tubular design. Means may be provided between the cap 16 and the seal element 17 for coupling purposes, such as coupling parts of a coupling mechanism, for example. These coupling parts may be provided in the form of projections 19, 20 on the cap 16 extending out from certain regions of its internal circumference, for example. The seal element 17 may also have a shoulder 21 extending radially beyond the sealing surface 18 which projects into the receptacle chamber of the cap 16 defined between the projections 19, 20. To facilitate assembly and improve the fixture of the shoulder 21 between the two projections 19, 20, an additional retaining ring 22 may be provided and disposed between the shoulder 21 and the projection 20, disposed in this instance on the side of the cap 16 facing away from the receptacle 5. The retaining ring 22, which in this instance is of an annular shape, has an end-to-end orifice in the region of the longitudinal axis 6 which provides access to the seal element 17t. In a manner known per se, in order to fill the receptacle chamber 13 of the receptacle 5, the seal element 17 may be pierced by means of a cannula, although this will not be described, thereby establishing a flow connection between the cannula and the receptacle chamber 13. This has long been known and a more detailed description will therefore not be given.

Providing the projection 19 between the shoulder 21 projecting out beyond the sealing surface 18 and the open end face of the receptacle 5 prevents the shoulder 21 from sticking or firmly adhering directly on the end face of the receptacle 5.

In its non-deformed initial state, the separating element 14 is predominantly spherical in terms of its three-dimensional shape. By predominantly spherical in this respect is meant that it corresponds to the basic three-dimensional shape of a sphere, and deviations from the spherical shape caused by production or manufacturing methods in terms of manufacturing accuracy are within the standard tolerance range.

With this receptacle device 1, the separating element 14 is disposed in the region of the second end 8 closed by the base 9 before filling the receptacle chamber 13 with the mixture to be separated 2. As may be seen from the diagram in FIG. 1, the separating element 14 sits at least against the internal face 11 of the side wall 10 in a contact plane 23 oriented perpendicular to the longitudinal axis 6. Furthermore, an external diameter 24 of the separating element 14 when the separating element 14 is in the non-deformed initial state is bigger than a cross-sectional dimension 25 of the receptacle chamber 13 in the contact plane 23. Due to these dimensional differences, the separating element 14 is held positioned in a pre-tensioned standby position against the internal face 11 of the side wall 10. Depending on the selected dimensional difference, a contact area 26 of the separating element 14 may range from a linear contact or abutment to an approximately cylindrical contact area 26 extending on either side of the contact plane 23.

For example, the external diameter 24 of the separating element 14 in its non-deformed initial state is within a range of between 1% and 10%, in particular between 2% and 5%, bigger than the cross-sectional dimension 25 of the receptacle chamber 13 in the contact plane 23 or in the region of the contact plane 23. The elastically deformable separating element 14 may have a Shore A hardness selected from a range of between 7 Shore A and 20 Shore A, in particular between 9 Shore A and 12 Shore A. By particular preference, the Shore hardness may have a value of 10 Shore A.

If the receptacle device 1 has to be filled with blood as the mixture to be separated 2, for example, the two phases 3, 4 have a different density or density value from one another. In a known manner, the lighter phase 3 may have a density of between 1.02 kg/m³ and 1.03 kg/m³ and the heavier phase 4 a density of between 1.05 kg/m³ and 1.09 kg/m³. The density values of the two phases 3, 4 for males and females are basically identical but the quantity ratio is different. The density of whole blood is usually subject to fluctuation because the proportions of the two phases are not always identical. This also depends on gender and in the case of males, for example, the lighter phase 3 represents a proportion by volume of whole blood in a range of between 50% and 60%. In the case of females, the proportion by volume of whole blood lies within a range of between 55% and 70%. This also results in a difference in the height of the position of the interface between the two phases 3, 4, which therefore not only depends on the total filling quantity but also on the gender of the patient or donor.

To ensure that the separating element 14 floats and is disposed between the phases to be separated 3, 4 during the separation operation or separation of the mixture 2, the separating element 14 should have a density selected from a range of between 1.02 kg/m³ and 1.09 kg/m³, in particular between 1.03 kg/m³ and 1.04 kg/m³. Accordingly, the density value of the separating element 14 or its material lies between the density values of the lighter phase 3 and the heavier phase 4.

To make it easier to introduce the mixture 2 into the receptacle chamber 13 when the receptacle 5 is closed, the closed receptacle chamber 13 of the receptacle 5 may be at a pressure that is below atmospheric pressure in a known manner.

The three-dimensional shape of the receptacle 5 and the closure unit 15, in particular the cap 16, in the region of their exterior or external faces is preferably selected so that they conform to the usual standardized dimensions or three-dimensional shapes. This enables a standardized, subsequent sample analysis and automated removal of part quantities of the separated phases 3, 4 from the receptacle chamber 13. The internal face 11 of the side wall 10 merges into a base surface 28 formed by the base 9 in a transition region 27. The base 9 preferably has a spherical dome-shaped three-dimensional shape.

In this example of an embodiment, the internal face 11 of the side wall 10 has several different part sections 29, 30 as viewed in the axial direction. The elastically deformable separating element 14 preferably also lies tightly against the base surface 28 formed by the base 9 and sits against the base surface 28 formed by the base 9 without any gap. This is illustrated on a simplified basis in FIG. 1.

As may best be seen from FIG. 2, a first part section 29 of the internal face 11 adjacent to the first, open end 7 is virtually cylindrical by reference to the longitudinal axis 6. An axial length of the first part section 29 corresponds at least to a longitudinal extension of the sealing surface 18 of the seal element 17 of the closure unit 15 inserted in the receptacle chamber 13. However, this axial longitudinal extension of the first part section 29 may also be longer than this, for example twice or three times the longitudinal extension of the sealing surface 18.

A second part section 30 of the internal face 11 may also be of a virtually cylindrical design by reference to the longitudinal axis 6. The second part section 30 may extend from the transition region 27 between the internal face 11 and base 9 in the direction towards the first open end 7 across an axial length which corresponds to at least 50%, preferably 60%, of the filling volume of the receptacle chamber 13. However, it would also be possible for the second part section 30 to extend continuously from the transition region 27 between the internal face 11 and base 9 as far as the first part section 29 in the region of the first open end 7. As may also be seen, the first part section 29 has a diameter which is bigger than that of the second part section 30. The expression virtually cylindrical should also be understood as meaning having a very slight conicity, the reason for this being that it ensures perfect demolding of the receptacle 5 during manufacture by injection casting.

In order to obtain a transition between the part sections 29, 30 of different diameters, a transition section 31 which tapers conically at its end facing the base 9 may be provided or disposed adjoining the first part section 29.

As may also be seen in FIG. 2 in the region of the closed second end 8, indicated by broken lines, at least one passage 32 may be provided or disposed between the base 9 and the second part section 30 of the internal face 11. This passage 32 or passages may be provided either in the form of recesses in the internal face 11 of the side wall 10 and/or by means of webs or projections extending in the radial direction around the internal face 11 in the direction towards the longitudinal axis 6, for example. The purpose of the passage or passages 32 is to provide a flow passage between part chambers of the receptacle chamber 13 disposed on either side of the contact plane 23 when the separating element 14 is disposed in the receptacle chamber 13 in the standby position in the region of the base 9. This means that when the receptacle chamber 13 is at a pressure below atmospheric pressure, the part chamber disposed between the contact plane 23 and base 9 can also be reduced to a lower pressure. This might be the case if the separating element 14 was introduced into the receptacle chamber 13 without evacuating it beforehand and the air or a partial quantity of it contained in the receptacle chamber 13 is included in the part chamber adjacent to the base 9.

In order to move the separating element 14 into its standby position in the base region before closing the receptacle chamber with the closure unit 15 and before filling the receptacle chamber with the mixture 2, there is a choice of different processing methods or operations for producing the receptacle device 1.

For example, in a known manner, the receptacle 5 can be made from a plastic material in an injection casting process, in which case it has the first and second ends 7 spaced apart from one another in the direction of the longitudinal axis 6. The receptacle chamber 13 is bounded by the base 9 and the side wall 10. At least in the region of the side wall 10, the receptacle 5 is made solely and exclusively from the plastic material, which also forms the internal face of the receptacle 5. The side wall 10 with its internal face therefore affords a stable and firm contact face for the separating element 14. A coating of material may also be provided on the internal face of the side wall 10 and optionally the base 9. The separating element 14 is made continuously and entirely from the elastically deformable material described above and is then placed in the receptacle chamber 13. By continuously and entirely is meant that the separating element 14 is made solely and hence entirely from a single material. Different assembly options and methods will be described below.

The essential point is that the separating element 14 has a predominantly spherical three-dimensional shape. Furthermore, before filling the receptacle chamber 13 with the mixture to be separated 2, the separating element 14 is moved to the region of the closed second end 8 incorporating the base 9 where it is held in position due to the dimensional differences in the pre-tensioned standby position described above. Accordingly, in the region of the contact plane 23 and the longer contact area 26 disposed in the axial direction, the external face of the separating element 14 sits in contact with the internal face 11 of the side wall 10.

FIG. 3 is a simplified illustration showing a first assembly option and a method for introducing the separating element 14 into the region of the base 9 of the receptacle 5. This approach may be construed as an independent solution in its own right, the same reference numbers and component names being used to denote parts that are the same as those described in connection with FIGS. 1 and 2 above. To avoid unnecessary repetition, reference may be made to the more detailed description of FIGS. 1 and 2 above.

In this instance, before being introduced into the receptacle chamber 13, the non-deformed spherical separating element 14 is inserted in a separate fitting tube 33 illustrated on a simplified basis. The fitting tube 33 has an external dimension that is smaller than a clear internal dimension of the receptacle chamber 13 so that it can be pushed unhindered into the receptacle chamber 13. Once the separating element 14 has been inserted in or introduced into the fitting tube 33 by elastic deformation, the fitting tube 33 is pushed or moved into the receptacle chamber 13 to the degree that its end face 34 is disposed adjacent to the base 9. Once the fitting position has been reached, the separating element 14 can then be pushed out of the fitting tube 33.

Due to selected dimensional differences or differences in diameter between the fitting tube 33 and the side wall 10 of the receptacle 5, the quantity of air still disposed between the base 9 and the separating element 14 is able to escape, for example as the separating element 14 is pushed out of the fitting tube 33. It escapes from the receptacle chamber 13 between the internal face 11 of the side wall 10 and the external face of the fitting tube 33, for example. This prevents any ambient air from being included in the part chamber of the receptacle chamber

13 disposed between the contact plane 23 and the base 9.

Furthermore, it would also be possible to provide several separating elements 14 inside the fitting tube 33 at the same time, one after the other, so that one of the separating elements 14 can be placed respectively in several receptacles one after the other. This enables fitting times to be reduced because another separating element 14 does not have to be inserted in the fitting tube 33 after every individual positioning operation.

FIG. 4 illustrates a second assembly option and method for introducing the separating element

14 into the region of the base 9 of the receptacle 5, illustrated on a simplified basis. This approach may also be construed as an independent solution in its own right, the same reference numbers and component names being used to denote parts that are the same as those described in connection with FIGS. 1 to 3 above. To avoid unnecessary repetition, reference may be made to the more detailed description of FIGS. 1 to 3 above.

Based on this approach, illustrated in a simplified basis, the separating element 14 is pierced by means of a hollow assembly pin 35, during which the assembly pin 35 completely penetrates the separating element 14. However, the elastic material of the separating element 14 should be pierced in such a way that the material of the separating element 14 is merely should merely pierced in the region of the assembly pin 35 but no part of the separating element 14 is severed. This might be described as spearing the separating element 14 on the hollow assembly pin 35. Once this has been done, the separating element 14 is moved to the open end 7 together with the assembly pin 35 and by means of the assembly pin 35 and placed in a sealing arrangement in contact with the internal face 11 of the side wall 10. The assembly pin 35 is then pushed by its end face 36 facing the base 9 into the receptacle chamber 13 to the degree that the end face 36 is disposed adjacent to the base 9. Due to the dimensional differences between the separating element 14 and the two part sections 29, 30 described above, the separating element 14 can be held pre-positioned in the region of the transition section 31 and in the part section 30 of the internal face 11 and the first part section 29 directly adjoining it in the direction towards the base 9. Care must be taken to ensure that the separating element 14 sits so that it affords a sealing contact all around.

If the second part section 30 starting from the transition region 27 or contact plane 23 is of a virtually cylindrical design across only a part section of the axial extension to the transition section 31, the separating element 14 can be pushed farther into the receptacle chamber 13 in the direction towards the base 9. When the separating element 14 lies in contact with the internal face 11 of the side wall 10 and is providing a seal all around, the part chamber of the receptacle chamber 13 sealed between the base 9 and the separating element 14 can be placed at a pressure below atmospheric pressure via the hollow assembly pin 35 extending through the separating element 14. This is done by means of a vacuum pressure unit 37, illustrated on a simplified basis.

By creating a pressure difference between the part chamber of the receptacle chamber 13 sealed by the separating element 14 and the external environment, the separating element 14 is moved along the assembly pin 35 in a sliding movement to the second end 8 of the receptacle 5 closed by the base 9.

Once the separating element 14 has reached the standby position in the receptacle chamber 13 as described above, the assembly pin 35 can be pulled out of the separating element 14. When pulling the assembly pin 35 out of the separating element 14, it may also be helpful to use a positioning element 38, which is likewise tubular for example, and holds the separating element 14 in the standby position until the assembly pin 35 has been removed from the separating element 14. The fitting tube 35 and positioning element 38 can then be pulled out of the receptacle 5 together.

If desired, before closing the open first end 7 by means of the closure unit 15, the pressure in the receptacle chamber 13 can then be reduced to a level lower than that of the external ambient pressure. This feature has long been known and makes filling of the receptacle chamber

13 with the mixture 2 to be contained in it easier and more reliable. This is of particular advantage in the case of blood samples.

FIG. 5 is a simplified illustration of a third assembly option and method for inserting the separating element 14 into the region of the base 9 of the receptacle 5. This approach may also be construed as an independent solution in its own right, the same reference numbers and component names being used to denote parts that are the same as those described in connection with FIGS. 1 to 4 above. To avoid unnecessary repetition, reference may be made to the more detailed description of FIGS. 1 to 4 above.

In the case of the approach and method for inserting the separating element 14 in the receptacle chamber 13 of the receptacle 5 described here, before closing the receptacle 5 by means of the closure unit 15, the area surrounding the receptacle 5 is reduced to a pressure below atmospheric pressure. This may take place in a chamber 39, for example, as indicated by broken lines. The pressure inside the chamber 39 can be reduced by means of a schematically indicated vacuum pressure unit 37, for example.

Reducing the pressure inside the chamber 39 results in a prevailing pressure that is lower than that of the external environment. Since the first open end 7 of the receptacle 5 has not yet been closed by means of the closure unit 15, this reduced pressure also prevails in the receptacle chamber 13. In the state of reduced pressure, the separating element 14 is then moved into the first open end 7 of the receptacle 5 following a displacement path indicated by “A” from where it is moved in the direction towards the base 9 to the degree that the separating element 14 sits in contact with the internal face 11, preferably in a sealing arrangement. This intermediate position of the separating element 14 is indicated by broken lines.

The receptacle 5 is then exposed to at least the external ambient pressure and because of the pressure difference created between the receptacle chamber 13 and the external ambient pressure the separating element 14 is moved along the other indicated displacement path “B” to the second end 8 closed by the base 9. The resultant standby position is indicated by dotted-dashed lines.

Exposure of the receptacle 5 with the separating element 14 pre-positioned in it may also take place inside the chamber 39, for example. For example, it is possible for the pressure difference to be created by only the external ambient pressure. However, another option would be to create a pressure inside the chamber 39 that is higher than the ambient pressure so that the pressure difference between the reduced pressure in the receptacle chamber 13 and the pressure prevailing in the chamber 39 is increased. This might then be described as an overpressure. Due to the pressure difference created between the receptacle chamber 13 closed by the separating element 14 and the area around the separating element 14, the latter is moved to the second end 8 closed by the base.

Since, prior to inserting the separating element 14 in the evacuated receptacle chamber 13 in its sealing arrangement, no or almost no residual quantity of air is contained in it, there is also no risk of a residual quantity of air being included in the part chamber or part section of the receptacle chamber 13 between the contact plane 23 or contact area 26, the base surface 28 and the separating element 14.

When the separating element 14 is in its standby position in the region of the base 9, the receptacle chamber 13 of the receptacle 5 can in turn be reduced to a pressure below atmospheric pressure and the closure unit 15 can then be placed on the receptacle 5 in the region of its open end 7, thereby sealing it.

In this respect, it should be pointed out that the receptacle 5 itself may be made from a range of different materials. It is preferable to use plastics, although glass might also be used as the material. The plastic may be selected, for example, from the group comprising polyethylene terephthalate (PET), polypropylene (PP), polyethylene (PE), polystyrene (PS), high-density polyethylene (HD-PE, acrylonitrile butadiene styrene copolymers (ABS), ultra-high molecular polyethylene with a very high molar mass (UHMW-PE), polycarbonate (PC), polyamide (PA), polyoxymethylene (POM).

Furthermore, it is also possible, before inserting or introducing the separating element 14 into the receptacle chamber 13, to coat the internal face 11 of the side wall 10 with a coating composition. This coating might be used, for example, to treat or act on the mixture 2 to be contained in the receptacle chamber 13. This might be used, for example, to prevent or initiate the coagulation of blood. In order to separate plasma as a lighter phase 3, an anti-coagulant could be applied to at least certain regions of the internal face 11, for example.

Furthermore, however, it would also be possible for the coating to be formulated in such a way that the sliding behavior of the separating element 14 is improved for the operation of moving it to the standby position and/or for the subsequent splitting or separation process. The coating might also reduce or improve sliding friction between the separating element 14 and internal face 11 of the side wall 10, for example, and increase static friction.

Independently of the above, however, the separating element 14 may also be provided with a coating in order to make it more difficult for constituents of the mixture 2 to adhere or prevent it altogether. A nano-coating or similar could be used for this purpose, for example.

Once a receptacle device 1 has been fully prepared in the manner described above, the receptacle chamber 13 can be filled with the mixture 2, for example by taking a blood sample. Having been filled with the mixture 2, it can then be subjected to a centrifugal force that will act on it. As a result of the centrifugal forces, the mixture 2 is separated into the lighter phase 3 having the lower density and the heavier phase 4 having the higher density. During this separation operation, the constituents of the heavier phase 4 are moved closer to the base 9 and the constituents of the lighter phase 3 float on those of the heavier phase 4. Under the effect of the centrifugal force, the separating element 14 is also elastically deformed to the degree that certain regions of the separating element 14 are moved from the standby position held pretensioned in contact with the internal face 11 of the side wall 10 to a position at a distance apart from the side wall. At least one flow passage is therefore created between the separating element 14 and the internal face 11 of the side wall 10 of the receptacle 5. By creating this flow passage, the constituents of the heavier phase 4 can now pass through and move into the region of the base 9. Due to the fact that the density of the separating element 14 is selected so that it is lower than that of the constituents of the heavy phase 4, the separating element 14 floats on the constituents of the heavier phase 4. Due to this floating, the still elastically deformed separating element 14 automatically moves into the separation plane created between the two mutually separated phases during the process of separating the mixture 2.

Once the two phases 3, 4 have been sufficiently separated from one another, the centrifugal force acting on them can be reduced and then finally stopped. As the centrifugal force is at least partially removed or reduced, the flow passage created between the separating element 14 and the internal face 11 of the side wall 10 is closed to form a seal by the separating element 14 as it elastically rebounds.

Due to the dimensional differences between the separating element 14 and the second part section 30 of the receptacle 5 described above, the separating element 14 also sits in contact with the internal face 11 of the side wall 10 providing a seal all the way round.

In order to obtain sufficient separation of the mixture 2 into its two phases 3, 4, an appropriate time must be selected for the centrifugal force to have its effect. The time may be a few minutes and it is preferable to select a time of at least 10 minutes.

The embodiments illustrated as examples represent possible variants of the receptacle device 1, and it should be pointed out at this stage that the invention is not specifically limited to the variants specifically illustrated, and instead the individual variants may be used in different combinations with one another and these possible variations lie within the reach of the person skilled in this technical field given the disclosed technical teaching.

Furthermore, individual features or combinations of features from the different examples of embodiments described and illustrated may be construed as independent and inventive solutions of the invention.

The objective underlying the independent inventive solutions may be found in the description.

All the figures relating to ranges of values in the description should be construed as meaning that they include any and all part-ranges, in which case, for example, the range of 1 to 10 should be understood as including all part-ranges starting from the lower limit of 1 to the upper limit of 10, i.e. all part-ranges starting with a lower limit of 1 or more and ending with an upper limit of 10 or less, e.g. 1 to 1.7, or 3.2 to 8.1 or 5.5 to 10.

Above all, the individual embodiments of the subject matter illustrated in FIGS. 1, 2; 3; 4; 5 constitute independent solutions proposed by the invention in their own right. The objectives and associated solutions proposed by the invention may be found in the detailed descriptions of these drawings.

For the sake of good order, finally, it should be pointed out that, in order to provide a clearer understanding of the structure of the receptacle device 1, it and its constituent parts are illustrated to a certain extent out of scale and/or on an enlarged scale and/or on a reduced scale.

### LIST OF REFERENCE NUMBERS

1 Receptacle device 32 Passage
2 Mixture 33 Fitting tube
3 Lighter phase 34 End face
4 Heavier phase 35 Assembly pin
5 Receptacle 36 End face
6 Longitudinal axis 37 Vacuum pressure unit
7 First end 38 Positioning element
8 Second end 39 Chamber
9 Base
10 Side wall
11 Internal face
12 External face
13 Receptacle chamber
14 Separating element
15 Closure unit
16 Cap
17 Seal element
18 Sealing surface
19 Projection
20 Projection
21 Shoulder
22 Retaining ring
23 Contact plane
24 External diameter
25 Cross-sectional dimension
26 Contact area
27 Transition region
28 Base surface
29 First part section
30 Second part section
31 Transition section

1. Receptacle device (1) for separating a mixture (2), in particular blood, into a lighter phase (3) with a lower density and a heavier phase (4) with a relatively higher density, comprising a receptacle (5) having a first end (7) and a second end (8) spaced apart from one another in the direction of a longitudinal axis (6), the first end (7) being open and the second end (8) being closed by a base (9), and a side wall (10) with an internal face (11) and an external face (12) extends between the first and the second end (7, 8), and the side wall (10) and base (9) bound a receptacle chamber (13), a separating element (14) which is disposed in the receptacle chamber (13) and is made entirely and continuously from an elastically deformable material, the density of the separating element (14) lying between that of the phases to be separated (3, 4), a removable closure unit (15) which closes the first, open end (7) of the receptacle (5), wherein in its non-deformed initial state, the separating element (14) is of a predominantly spherical three-dimensional shape and is disposed in the region, of the second end (8) closed by the base (9) before the receptacle chamber (13) is filled with the mixture to be separated (2) and sits in contact with the internal face (11) of the side wall (10) at least in a contact plane (23) oriented perpendicular to the longitudinal axis (6), and an external diameter (24) of the separating element (14) is selected so as to be bigger in its non-deformed initial state than a cross-sectional dimension (25) of the receptacle chamber (13) in the contact plane (23) and the separating element (14) is thus held positioned against the internal face (11) of the side wall (10) in a pre-tensioned. standby position.

2. Receptacle device (1) according to claim 1, wherein the external diameter (24) of the separating element (14) in its non-deformed initial state is in a range of between 1% and 10%, in particular between 2% and 5%, bigger than the cross-sectional dimension (25) of the receptacle chamber (13) in the contact, plane (23).

3. Receptacle device (1) according to claim 1, wherein the separating element (14) has a Shore A hardness selected from a range of between 7 Shore A and 20 Shore A, in particular between 9 Shore A and 12 Shore A, particularly preferably 10 Shore A.

4. Receptacle device (1) according to claim 1, wherein the separating element (14) has a density selected from a range of between 1.02 kg/m³ and 1.09 kg/m³, in particular between 1.03 kg/m³ and 1.04 kg/m³.

5. Receptacle device (1) according to claim 1, wherein the closed receptacle chamber (13) of the receptacle (5) is reduced to a pressure below atmospheric pressure.

6. Receptacle device (1) according to claim 1, wherein a first part section (29) of the internal face (11) directly adjacent to the first, open end (7) is of a virtually cylindrical design by reference to the longitudinal axis (6).

7. Receptacle device (1) according to claim 6, wherein an axial length of the first part section (29) corresponds to at least a longitudinal extension of a sealing surface (18) of a seal element (17) of the closure unit (15) inserted in the receptacle chamber (13).

8. Receptacle device (1) according to claim 1, wherein a second part section (30) of the internal face (11) is of a virtually cylindrical design by reference to the longitudinal axis (6) and this second part section (30) extends from a transition region (27) between the internal face (11) and base (9) in the direction towards the first open end (7) across an axial length which corresponds to at least 50%, preferably 60%, of the filling volume of the receptacle chamber (13).

9. Receptacle device (1) according to claim 8, wherein the second part section (30) extends from the transition region (21) between the internal face (11) and base (9) as far as the first part section (29).

10. Receptacle device (1) according to claim 6, wherein the first part section (29) has a diameter which is bigger than that of the second part section (30).

11. Receptacle device (1) according to claim 6, wherein a conically tapering transition section (31) adjoins the first part section (29) on the side facing the base (9).

12. Receptacle device (1) according to claim 1, wherein at least one passage (32) is provided or disposed between the base (9) and the second part section (30) of the internal face (11) which connects part chambers of the receptacle chamber (13) disposed, on either side of the contact plane (23) when the separating element (14) is disposed in the receptacle chamber (13).

13. Method of providing a receptacle device for separating a mixture (2), in particular blood, into a lighter phase (3) with a lower density and a heavier phase (4) with a relatively higher density, whereby a receptacle (5) having a first end (7) and second end (8) spaced apart from one another in the direction of a longitudinal axis (6) is provided, the first end (7) being open and the second end (8) being closed by a base (9), and a side wail (10) with an internal face (11) and an external face (12) extends between the first and the second end (7, 8), the side wall (10) and the base (9) bounding a receptacle chamber (13), a separating element (14) made entirely and continuously from an elastically deformable material and disposed in the receptacle chamber (13), and the density of the separating element (14) is selected so that it is between that of the phases (3, 4) to be separated, and the first open end (7) of the receptacle (5) is closed by means of a removable closure unit (15), wherein the separating element (14) is of a predominantly spherical three-dimensional shape and the separating element (14) is moved into the region of the second end (8) closed by the base (9) before the receptacle chamber (13) is filled with the mixture to be separated (2) and thus sits in contact with the internal face (11) of the side wall (10) in a contact plane (23) oriented perpendicular to the longitudinal axis (6), and when producing the separating element (14), an external diameter (24) of the separating element (14) is made so as to be bigger in its non-deformed initial state than a cross-sectional dimension (25) of the receptacle chamber (13) in the contact, plane (23) and the separating element (14) is thus held positioned against the internal face (11) of the side wall (10) in a pre-tensioned standby position.

14. Method according to claim 13, wherein before being inserted in the receptacle chamber (13), the separating element (14) is introduced into a fitting tube (33) having an external dimension that is smaller than a clear internal dimension of the receptacle chamber (13), after which the fitting tube (33) is moved into the receptacle chamber (13) to the degree that its end face (34) is disposed adjacent to the base (9) and the separating element (14) is then pushed out of the fitting tube (33).

15. Method according to claim 13, wherein the separating element (14) is pierced by a hollow assembly pin (35) and the separating element (14) is then placed against the internal face (11) of the side wall (10) in a sealing arrangement and the assembly pin (35) is pushed into the receptacle chamber (13) to the degree that its end face (36) is disposed adjacent to the base (9), and the sealed receptacle chamber (13) between the base (9) and the separating element (14) is reduced by means of the hollow assembly pin (35) to a pressure below the external ambient pressure, and due to the pressure difference created between the receptacle chamber (13) and the external environment, the separating element (14) is moved on the assembly pin (35) in a sliding movement to the second end (8) closed by the base (9).

16. Method according to claim 13, wherein before closing the receptacle (5) by means of the closure unit (15), the area surrounding the receptacle (5) is reduced to a pressure below the external ambient pressure, and the separating element (14) is placed in the first open end (7) of the receptacle (5) and moved so that it sits in contact with the internal face (11), after which the receptacle (5) is exposed to a higher pressure, in particular to the external ambient pressure, and due to the pressure difference created between the receptacle chamber (13) and the area surrounding the separating element (14) the latter is moved to the second end (8) closed by the base (9).

17. Method according to claim 13, wherein the receptacle chamber (13) of the receptacle (5) is reduced, to a pressure below atmospheric pressure and the. closure unit (15) is then placed on the receptacle (5).

18. Method for separating a mixture (2), in particular blood, into a lighter phase (3) with a lower density and a heavier phase (4) with a relatively higher density, using a receptacle device according to claim 1, whereby the receptacle chamber (13) closed off from the external environment by the closure unit (15) is filled with the mixture to be separated (2), in particular blood, and the contained mixture (2) is then subjected to a centrifugal force that acts on it and the mixture (2) is thus separated into the phase (3) with the lower density and the phase (4) with the higher density, and under the effect of the centrifugal force the separating element (14) is elastically deformed to the degree that certain regions of the separating element (14) are moved from the standby position held pre-tensioned in contact with the internal face (11) of the side wall (10) to a position at a distance from the side wall (10), and a flow passage is thus created between the separating element (14) and the internal face (11) of the side wall (10) of the receptacle (5), and the elastically deformed separating element (14) automatically moves into a separation plane created between the two mutually separated phases (3, 4) during the process of separating the mixture (2), and after at least partially removing the centrifugal force, the flow passage created between the separating element (14) and the internal face (11) of the sidewall (10) is closed by the separating element (14) as it elastically rebounds forming a seal.

19. Method according to claim 18, wherein the mixture (2) is subjected to centrifugal force for a time of at least 10 min.
